(19) United States
(12) Patent Application Publication
(10) Pub. No.: US 2024/0041506 A1
(43) Pub. Date: Feb. 8, 2024

(54) BENDING THE PROBES OF DEPTH GAUGES

(71) Applicant: EDGE SURGICAL, INC., Chicago, IL (US)

(72) Inventors: Robert F. Rioux, Ashland, MA (US); Alan Rabideau, Cary, NC (US)

(21) Appl. No.: 18/382,772

(22) Filed: Oct. 23, 2023

Related U.S. Application Data
(63) Continuation-in-part of application No. 17/971,096, filed on Oct. 21, 2022.
Application No. 17/971,096 is a continuation of application No. 16/810,320, filed on Mar. 5, 2020, now Pat. No. 11,504,169.
(60) Provisional application No. 62/937,526, filed on Nov. 19, 2019.
(60) Provisional application No. 62/816,536, filed on Mar. 11, 2019.

(51) Int. Cl. A61B 17/70; A61B 90/00; A61B 17/17; A61B 17/58

(52) U.S. Cl. CPC A61B 17/7092; A61B 90/06; A61B 90/36; A61B 17/1728; A61B 17/58; A61B 2560/0431; A61B 2562/02; A61B 2562/166; A61B 2090/062; A61B 2090/036; A61B 2090/0811; A61B 2562/0247

(57) ABSTRACT

A device for bending a probe of a depth gauge can be used on the depth gauge's probe before the depth gauge is sealed in sterile packaging, and another device for bending the depth gauge's probe can be used after the depth gauge is removed from its sterile packaging.

### CROSS-REFERENCE TO RELATED APPLICATIONS

This application is a continuation-in-part of, and claims the benefit of, and priority to, U.S. Non-Provisional application Ser. No. 17/971,096, filed on Oct. 21, 2022. U.S. Non-Provisional application Ser. No. 17/971,096 is a continuation of U.S. Non-Provisional application Ser. No. 16/810,320, filed on Mar. 5, 2020. U.S. Non-Provisional application Ser. No. 16/810,320 is now U.S. Pat. No. 11,504,169, issued on Nov. 22, 2022. U.S. Pat. No. 11,504,169 claims the benefit of, and priority to, U.S. Provisional Application No. 62/937,526, filed on Nov. 19, 2019, and 62/816,536, filed on Mar. 11, 2019. The entire contents of each of these four applications are incorporated herein by reference.

### TECHNICAL FIELD

The invention generally relates to devices for use in connection with medical procedures that involve fixing one or more implants (such as a surgical screw) into one or more bones of a patient.

### BACKGROUND INFORMATION

Orthopedics is a medical specialty concerned with the correction of deformities or functional impairments of the skeletal system, in particular the extremities and the spine, and associated structures, such as muscles and ligaments, of a human (or other type of mammal) patient. Some orthopedic surgical procedures require surgeons to secure a device to one or more bones of a patient. For example, in some procedures, a surgeon may span and secure one or more bones, or pieces of a single bone, using a bone plate and one or more fasteners, such as screws. Other bone-related surgical procedures, however, may not require a bone plate and may instead solely rely on the use of one or more screws to, for example, secure a transplanted tendon.

In any of a variety of bone-related surgical procedures, before an implant or plate, or simply a screw itself, can be attached to bone, an opening is typically drilled into that bone to accommodate the screw. With the opening or hole in place, the surgeon can more easily select a screw of the appropriate length. However, selecting a screw of appropriate length is critical. If the selected screw is too long, for example, the distal end of the screw may pass through the end of the drilled hole and cause damage to the bone and/or protrude entirely through the bone, which can have deleterious effects, such as damage to surrounding tissue and/or pain and discomfort, or more serious complications, for the patient. For example, in some instances, the bone may abut against soft tissues that may be harmed if the screw is too long and that may result in irritation of or damage to those soft tissues. Additionally, a screw that protrudes through the bone may be tactilely felt by the patient, may prevent soft tissues (e.g., tendons, ligaments, or muscles) from moving over the bone surface as intended, or may even pierce the skin which can lead to serious infection and/or other complications.

The selection of an appropriate length screw is particularly important in spinal fixation procedures, such as lumbar sacral fusion and the correction of spinal deformities such as scoliotic curves. As an example, a screw mounted in the pedicle portion of the human spine should not extend to a point where the screw contacts the spinal cord itself, an event that can cause irreparable nervous system damage including paralysis. Accordingly, the determination of a length of the hole is important for choosing a screw of the appropriate length.

During the act of drilling into a bone, the surgeon is typically capable of recognizing the resistance on the drill to determine when the drill has penetrated through the bone. Because the simple act of drilling does not provide an exact measurement of the depth of the bone itself, a depth gauge is commonly employed for directly measuring the depth of the hole from the top, drilling side of the bone to the bottom, opposite side of the drilled hole.

There are known tools to measure the depth of a through hole in bone or the depth of a bore in bone. Some such tools use a central probe member with a barb at a distal end, and they also use a sleeve or channel member. The probe is inserted into the drilled pilot through hole in the bone while the surgeon attempts to find the opposite surface with the distal barb. More specifically, the probe is inserted to a depth greater than the depth of the pilot hole so that the barb is beyond the opposite side of the bone with the pilot hole drilled through it, at which point the surgeon finds the surface by hooking the barb to the opposite side. The probe is received in the sleeve or channel member and may reciprocate relative thereto. The channel member has graduated markings along a portion of its length, typically in inches and/or millimeters. A marker is laterally secured to the probe such that, as the probe shifts relative to the channel, the marker indicates the relative shift between the probe and the channel. Accordingly, once the distal end of the probe has been secured to the opposite side of the bone, the channel member is shifted relative to the probe and toward the bone until the channel member abuts the surface of the bone. The depth gauge is then read by visually examining graduated markings indicated by the probe marker.

A user of such a known depth gauge can experience problems. As an initial point, the components of such a gauge typically are made of surgical-grade stainless steel, and the graduated markings are embossed therein. The brightness of the operating room lights on such a highly reflective surface can make the markings difficult to read. The markings commonly are in small increments, such as millimeters, and surgeons often have trouble differentiating between the markings, or noting partial increments. Reading such a gauge often requires carefully holding the depth gauge as the reading is taken, and a surgeon's effort to examine closely the reading may result in a loss of securement or purchase of the barb on the bone, thus necessitating a re-measurement and subsequent loss of time. Furthermore, proper reading of the markings requires a surgeon's eyes to be properly aligned with the markings. That is, a proper view of the measurement requires the surgeon to view the gauge from a lateral point of view so that the view of the probe marker aligned with the graduated markings is proper and not distorted by the surgeon's elevated, standing perspective. Therefore, it is often necessary for the surgeon to bend over while using these gauges to view an accurate reading. If the depth gauge is tilted to make the reading easier, the sleeve will shift relative to the probe, thus making the measurement inaccurate and possibly also causing the distal end/barb of the probe to become unsecured. In addition, removal of the depth gauge often causes the measurement to be lost. As the bone is essentially clamped, by light pressure, between the distal end of the channel member and the distal end/barb of the probe, it is often necessary to retract the channel member from the bone surface to extract the probe from the pilot hole. Another problem with using such a known depth gauge is that it is cleaned and reused in multiple subsequent orthopedic surgical procedures, but the cleaning process can be inadequate in properly sterilizing the depth gauge such that an infection can occur in a patient.

### SUMMARY OF THE INVENTION

A depth gauge that is superior to known depth gauges is a single-use, disposable depth gauge that can be used in orthopedic surgical procedures and that is known as the EDG® depth gauge. The EDG® depth gauge is by EDGe Surgical, Inc. of Chicago, Illinois (edgesurgical.com), and the EDG® device has a digital display that provides to a user a readout of the device's depth measurement on an OLED (organic light-emitting diode) display screen. EDGe Surgical, Inc. owns patents directed to depth gauges with digital displays including, for example, U.S. Pat. Nos. 7,895,767, 10,132,607, and 10,151,570, as well as the priority U.S. Pat. No. 11,504,169.

A depth gauge with a bone probe (such as any of the versions of a single-use, disposable depth gauge disclosed in priority U.S. Pat. No. 11,504,169, which is incorporated herein in its entirety) can have a portion of its bone probe in an off-axis configuration. For example, the probe of a depth gauge can be bent, curved, or angled at a certain point along the length of the probe. That bend, curve, or angle of the probe of the depth gauge can be made during the manufacturing or assembly process of the depth gauge, such that the depth gauge is delivered to the end user (for example, an orthopedic surgeon or a nurse) in sterile packaging with the bend already present in the probe of the packaged depth gauge. Alternatively, the depth gauge can be packaged and delivered to the end user with a straight or substantially straight probe and with a tool (also provided in the sterile packaging, or else in sterile packaging that is separate from the sterile packaging containing the depth gauge) for use by the end user to bend the probe of the depth gauge before the end user uses (and/or during the time the end user is using) the depth gauge in an orthopedic medical procedure on a patient. The tool used in the process of manufacturing and/or assembling the depth gauge to bend the probe of the depth gauge can be referred to as a manufacturing tool, and the tool designed for use by an end user of the depth gauge to bend the probe of the depth gauge can be referred to as an end-user tool.

An end user (such as an orthopedic surgeon) would be able to use the manufacturing tool to bend a straight or substantially straight probe of a depth gauge if the manufacturing tool was provided to the end user, and a person involved in the manufacturing and/or assembly of the depth gauge would be able to use the end-user tool to bend the probe of a depth gauge if the end-user tool was provided to that manufacturing-assembly person, but the manufacturing tool is designed specifically for use by the manufacturing-assembly person to bend the probe of the depth gauge before the finished depth gauge is sealed in sterile packaging, and the end-user tool is designed specifically for use by an end-user person to bend the probe of the depth gauge after the finished depth gauge is removed from sealed sterile packaging. While each of the manufacturing tool and the end-user tool is designed to be used by a person to bend a portion of a probe of a depth gauge, the end-user tool generally is simpler in its construction, and simpler to use, when compared to the manufacturing tool. The end-user tool is designed to be used when held in the hand of an end user of the depth gauge device (such as an orthopedic surgeon or a nurse in an operating room), and the manufacturing tool is designed to be placed on a desktop or workbench surface and used by a manufacturing and/or assembly person (such as a technician located in a medical device manufacturing and/or assembly facility or room).

Either of the tools can be used to bend the probe at one or more locations or areas along the longitudinal axis of the probe. The probe generally will start in a straight or substantially straight longitudinal configuration before one of the tools is used to place one or more bends at one or more locations or areas along the probe's length, but the probe already could have some curve or angle to it before the tool is used on the probe. Whether or not the probe already has some bend(s) to it before the tool is used on the probe, the probe typically will have some longitudinal flexibility to it. This flexibility of the probe provides a “feel” to the end user (for example, a surgeon) during examination of a hole in a bone with the probe, but the bend(s) created in the length of the probe by use of one of the tools provides the end user with even greater tactile feel or feedback when using a bent-probe depth gauge in a medical procedure.

The bend (or bends) in the probe gives (or give) the depth gauge device a feature that will allow an end user (such as a surgeon or a nurse) to feel very well the interior side wall(s) of a drilled hole in a bone of a patient (when the probe is inserted into the hole and the depth gauge is manually manipulated by the end user) and/or to feel very well when the distal end of the probe emerges from the opposite end of the hole and catches on the lip of that opposite-side exit aperture of the hole. The user's tactile experience when using the depth gauge in a medical procedure is enhanced when the probe of the depth gauge is bent off-axis by a certain angle (or any particular angle within a certain range of angles) or curve, and that is why it is important to provide the end user with a depth gauge having a pre-bent probe and/or to provide the end user with the ability to bend the probe of a depth gauge after the depth gauge is removed from its sterile packaging.

In accordance with one aspect of the invention, a method involves bending a probe of a depth gauge by disposing within a tool at least a portion of the probe of the depth gauge and then operating the tool to bend the probe at a location along a length of the portion of the probe disposed within the device. The bent-probe depth gauge is then removed from the tool and can be either packaged for delivery to an end user (such as an orthopedic surgeon or unit at a hospital or surgery center) or else used by the end user in a medical procedure on a patient.

In accordance with another aspect of the invention, a device is designed for use by an end user (before and/or during a medical procedure on a patient) to bend a probe of a depth gauge after the depth gauge is removed from sterile packaging. The device comprises a first member, a second member, and a connector portion. The first member has a free end, a length, and a width, with the length of the first member being greater than the width of the first member. The second member has a free end, a length, and a width, with the length of the second member being greater than the width of the second member. The first and second members are disposed opposite each other with space between the first and second members, where the space is for receiving at least a portion of the probe of the depth gauge. The connector portion is disposed opposite the free ends of the first and second members to connect the first and second members, and the connector portion is configured to allow the first and second members to move to increase or decrease the space between the first and second members. A bend is created in the probe at a location along a length of the portion of the probe received within the space when the end user causes the first and second members to move and decrease the space between the first and second members. The connector portion can define an aperture through which the portion of the probe of the depth gauge passes to occupy at least a portion of the space. The first member, second member, and connector portion of the device can be formed as a single piece by an injection molding process, and the single piece can be formed of a thermoplastic material.

In accordance with yet another aspect of the invention, a device is designed for bending a probe of a depth gauge before the depth gauge is sealed in sterile packaging (for later removal from that packaging in connection with a medical procedure to be performed on a patient). The device comprises a base and a lever. The base is configured to be disposed on a work surface, and the base defines a channel for receiving at least a portion of the probe of the depth gauge. The lever is pivotally coupled to the base to allow the lever to move with respect to the base when the base is disposed on the work surface. A bend is created in the probe at a location along a length of the portion of the probe received within the channel when the lever is moved toward the channel of the base. A person involved in the manufacturing and/or assembly of the bent-probe depth gauge can be the one to push down on the lever to move it toward the base's channel and thereby create the bend in the probe. The base of the device also can define a different channel for receiving at least a portion of a body or handle of the depth gauge. The device can include a hinge pin disposed through the lever and received within the base to allow the lever to move with respect to the base. The base also can include a sliding member movable from an open position to a closed position, where the portion of the probe is able to be received within the channel when the sliding member is in the open position and held in place in the channel when the sliding member is in the closed position. A bottom of the base can have four feet for contacting the work surface when the base is disposed on the work surface. The base of the device also can include a form member with a curved surface for creating the bend in the probe when the lever is moved toward the channel of the base, and the lever of the device can include a roller member for pushing on the location along the length of the portion of the probe received within the channel to cause the bend to occur at that location and according to the curved surface of the form member when the lever is moved toward the base's channel. The device can be formed of one or more metals, or the device can be formed of one or more alloys.

These and other aspects and details of devices and tools according to the invention will become clearer by referring to the following parts of this document, and various methods of making and using such tools and devices will become clearer as well. The entirety of this document illustrates tools, devices, and methods that embody the invention, but the invention is not limited only to the specific tools, devices, and methods disclosed herein. Various ideas and combinations that can be derived from the contents of this document (including both the text and the accompanying drawings) are to be considered as disclosed and included herein.

### BRIEF DESCRIPTION OF THE DRAWINGS

FIGS. 1-8 show an end-user tool for use by an end user (such as a surgeon or a nurse) to bend a portion of a probe of a depth gauge after the depth gauge and the tool are removed from sterile packaging. FIG. 1 is a side perspective view of the end-user tool, and FIG. 2 is a side view of the end-user tool. FIG. 3 is a top view of the end-user tool. FIG. 4 is a perspective view of the end-user tool and a depth gauge having a straight (or substantially straight) probe before that probe has been inserted within the end-user tool via an opening in the end-user. FIG. 5 is a perspective view of the end-user tool with a top arm or member of the tool moved upward away from a bottom opposite arm or member and with the depth gauge's probe disposed within the tool between the two arms or members. FIG. 6 is a perspective view of the end-user tool with the top and bottom opposing arms or members moving toward each other to contact at least a portion of the depth gauge's probe that is disposed within the tool between the two arms or members. FIG. 7 is a side view of the depth gauge after its probe has been bent by use of the end-user tool and after it has been withdrawn from the tool, and FIG. 8 is a side view of a distal portion of the probe to show the bend created in the depth gauge's probe by use of the end-user tool.

FIGS. 9-18 show a manufacturing tool for use by a person involved in the manufacturing and/or assembly of a depth gauge to bend a portion of a probe of the depth gauge before the depth gauge is placed in sterile packaging. FIG. 9 is a side perspective view of the manufacturing tool, and FIG. 10 is an exploded side perspective view of the manufacturing tool. FIG. 11 is a perspective view of the manufacturing tool and a depth gauge having a straight (or substantially straight) probe before a body of the depth gauge and the probe have been received within channels of the manufacturing tool. FIG. 12A is a perspective view showing the depth gauge's body and probe disposed above the channels of the manufacturing tool, and FIG. 12B is a different perspective view with the depth gauge's body and probe disposed with the channels of the manufacturing tool. FIGS. 13A and 13B show the use of a sliding lock feature of the manufacturing tool, to hold the depth gauge's probe with a channel of the manufacturing tool. FIGS. 14 and 15 show how a lever of the manufacturing tool can be moved downward toward a base of the manufacturing tool to create a bend in a distal portion of the depth gauge's probe. FIGS. 16A, 16B, and 16C show additional details about how the manufacturing tool creates a bend in the distal portion of the probe of the depth gauge. FIG. 17 is side view of the depth gauge after its probe has been bent by use of the manufacturing tool and after it has been withdrawn from the tool, and FIG. 18 is a side view of the distal portion of the probe to show the bend created in the depth gauge's probe by use of the manufacturing tool.

Each of these twenty-two drawings is referenced again and explained in more detail below.

### DETAILED DESCRIPTION

The invention relates to devices and methods for bending the probe of a depth gauge to create at least one bend in the probe of the depth gauge. The bent-probe depth gauge can be used (by, for example, an orthopedic surgeon, and before and/or during a medical procedure performed on a patient) to determine the depth of a bore hole or a through hole in a bone of the patient.

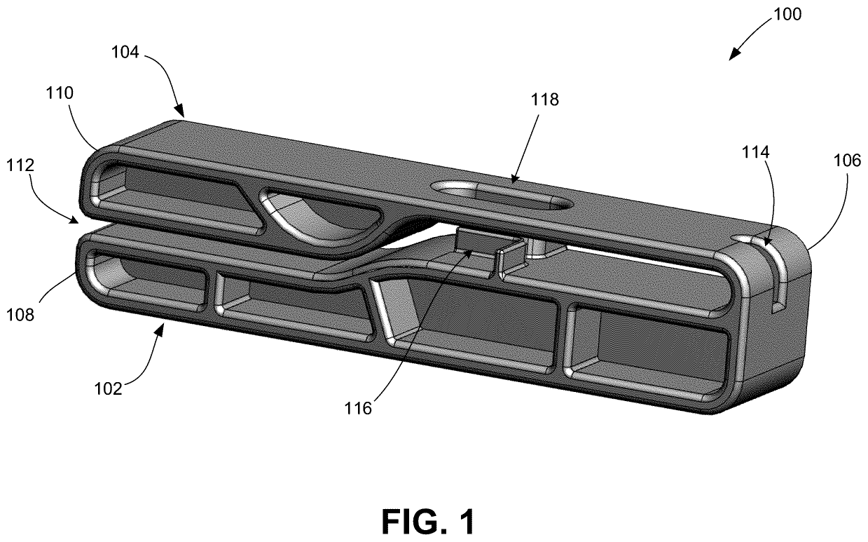

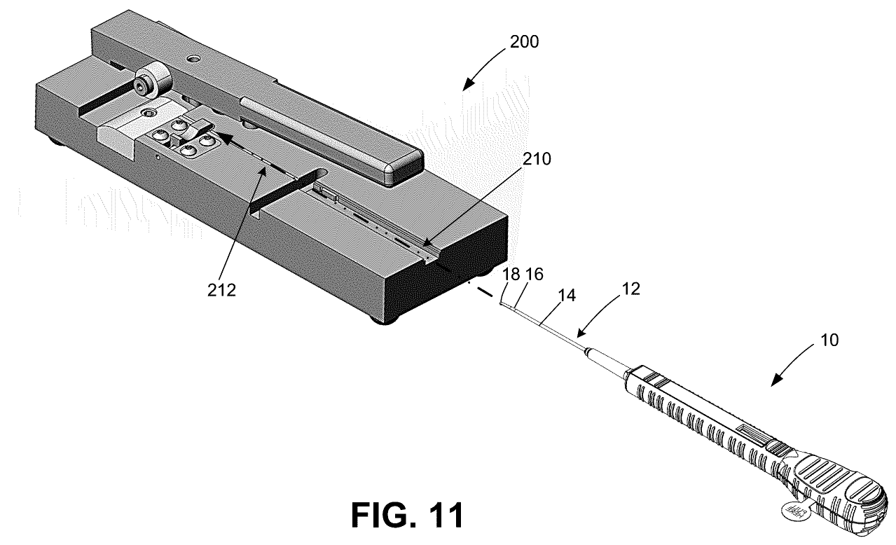

Turning to the drawings that are directed to the present invention, FIGS. 1-8 that accompany this text show a handheld probe bending tool 100 that is referenced herein as an end-user tool 100, and FIGS. 9-18 show a probe bending fixture 200 that is referenced herein as a manufacturing tool 200. The invention is not limited only to these two specific tools, and various combinations of the features and aspects of the described and illustrated tools and related methods are to be considered disclosed herein even if not specifically called out.

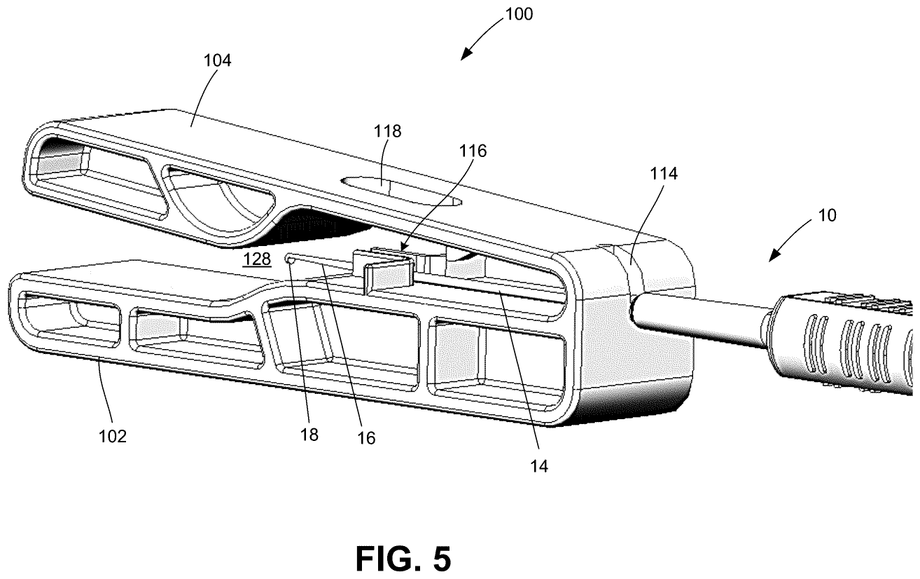

FIG. 1 shows the end-user tool 100 that is designed to be held in the hand(s) of a user (such as an orthopedic surgeon or an operating room nurse, for example) for use by the user (or some other person associated with the user) to bend a probe 12 (see FIG. 4) of a depth gauge 10 (see FIG. 4). The depth gauge 10 can be any of the single-use, disposable depth gauges disclosed in the priority U.S. Pat. No. 11,504,169 (which is incorporated herein in its entirety). Both the depth gauge 10 and the end-user tool 100 can be contained within sterile packaging (either together in the same sterile packaging, or else separately in two separate sterile packages) for the user or some person associated with or assisting the user to open to access the depth gauge 10 and the end-user tool 100 before and/or during a medical procedure on a patient. The end-user tool 100 is for a person to hold (in one or both hands) and use to bend the probe 12 of the depth gauge 10 after the depth gauge 10 and the end-user tool 100 are removed from sterile packaging.

The end-user tool 100 comprises a first arm or member 102 that has a free end 108, a length, and a width, where the length is greater than the width. The end-user tool 100 also comprises a second arm or member 104 that also has a free end 110, a length, and a width, where the length of the second member 104 also is greater than the width of the second member 104. The first and second members 102, 104 are disposed opposite each other with space 112 therebetween. At least a portion of the probe 12 of the depth gauge 10 can be received within the space 112. The end-user tool 100 also comprises a pivot or connector portion 106 that is disposed opposite the free ends 108, 110 of the first and second members 102, 104 to connect the first and second members 102, 104 and to allow the first and/or second members 102, 104 to move. The connector portion 106 is configured to allow the first and second members 102, 104 to move to increase or decrease the space 112 between the first and second members 102, 104. A bend is created in the probe 12 at a location along a length of the portion of the probe 12 that is received within the space 112 when the user manipulates the first and second members 102, 104 to cause the first and/or second members 102, 104 to move and decrease the space 112 between the first and second members 102, 104. It can be that the second member 104 is moved by the user toward the first member 102, that the first member 102 is moved by the user toward the second member 104, or that each of the first and second members 102, 104 moves toward the other one. Whatever movement of the first and/or second members 102, 104 that the user causes by the user's manipulation of the end-user tool 100 with the user's hand(s) can result in the space 112 between the first and second members 102, 104 decreasing such that a shaft 14 (see FIG. 4) of the probe 12 of the depth gauge 10 is bent by that movement of the first and/or second members 102, 104.

The connector portion 106 of the end-user tool 100 can define an aperture 114 through which the shaft 14 of the probe 12 of the depth gauge 10 can pass to occupy at least a portion of the space 112. The first member 102 of the end-user tool 100 can include a guide member 116 for retaining and aligning at least a portion of the shaft 14 of the probe 12 when placed through the aperture 114 and into the space 112. The second member 104 of the end-user tool 100 can define a viewing window 118 that is disposed such that a user of the end-user tool 100 can see the portion of the shaft 14 of the probe 12 that is retained and aligned by the guide member 116.

The first member 102, second member 104, and connector portion 106 of the end-user tool 100 can be formed as a single piece, and the single piece can be created by, for example, an injection molding process. Injection molding is a known manufacturing process for producing parts (including various medical devices) by injecting a molten material (such as a thermoplastic material) into a pre-formed mold and then letting the injected material cool and harden to thereby take the shape of the mold. A thermoplastic material can be fed into a heated barrel, mixed in that barrel by a helical screw turning within the barrel, and moved out of the barrel and into the cavity of the mold, where the injected material cools and hardens into the shape of the mold's internal cavity. The thermoplastic material used to create the single-piece end-user tool 100 can be, for example, polyethylene, polypropylene, polystyrene, polymethyl methacrylate, polyvinyl chloride, a polyamide, a polycarbonate, some other thermoplastic material, or a combination of two or more thermoplastic materials.

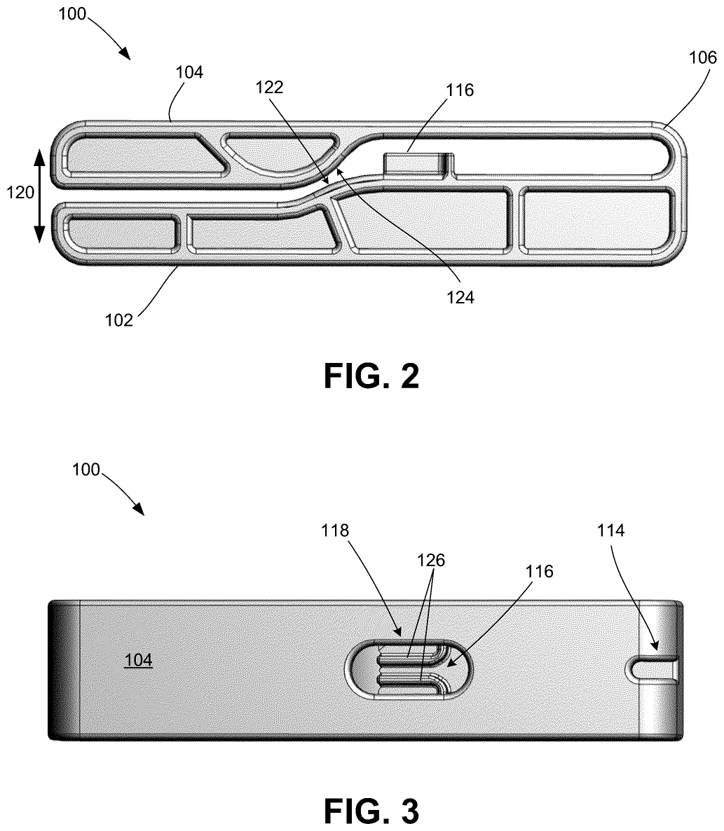

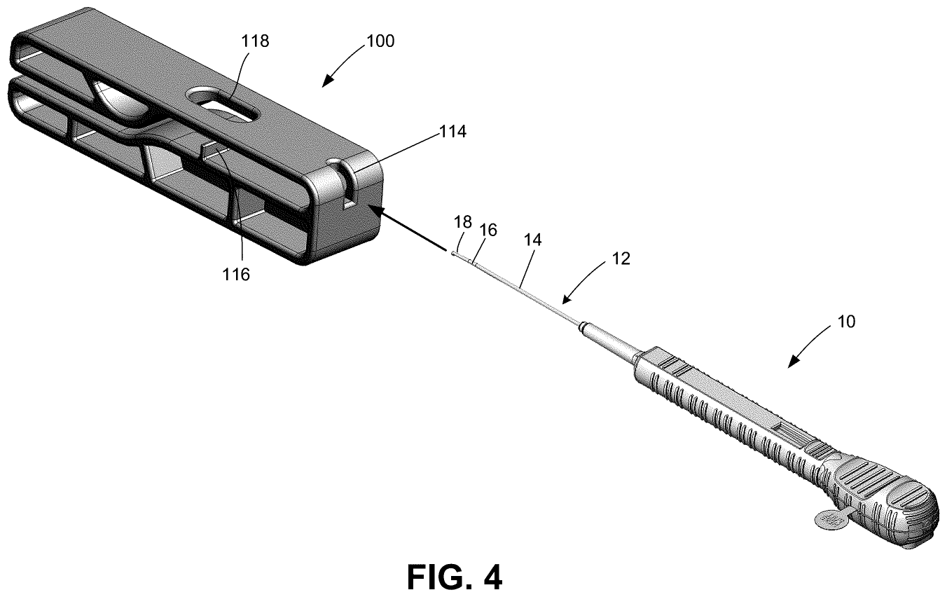

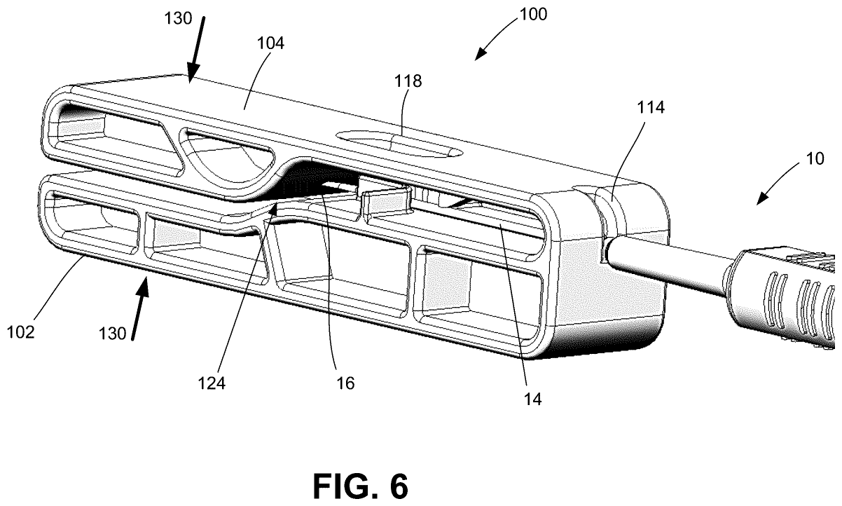

Referring now to FIGS. 2 and 3, the possible movement of the first and second members 102, 104 of the end-user tool 100 when a person holds the end-user tool 100 in his or her hand(s) is indicated by an arrow 120. When moved away from each other, the space 112 (see FIG. 1) is increased. When moved toward each other, the space 112 is decreased. The first member 102 has a bending surface 122, and the second member 104 also has a bending surface 124. These two bending surfaces 122, 124 are opposite each other and are located on the first and second members 102, 104, respectively, in a bending zone 128 (see FIG. 5) that is located within the space 112 between of the first and second members 102, 104 of the end-user tool 100. As shown in FIG. 3, the guide member 116 of the first member 102 includes a pair of ridges 126 that define a channel configured to receive the shaft 14 of the probe 12 of the depth gauge 10.

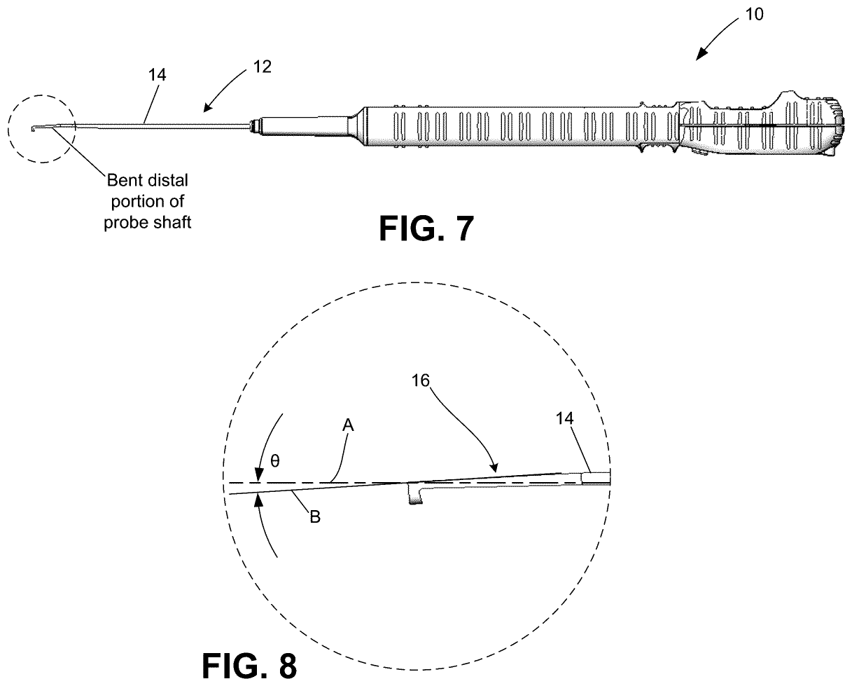

Turning now to FIGS. 5 and 6, the second member 104 is shown moved up away from the first member 102 (see FIG. 5) to create an increase in the space 112 between the first and second members 102, 104 and to show the location of the bending zone 128. A distal portion 16 of the shaft 14 of the probe 12 of the depth gauge 10 is located at or in the bending zone 128 when the shaft 14 is inserted fully into the space 112 of the end-user tool 100. The probe 14 includes a distal end 18 that defines a probing tip. The probing tip can be the probing tip that is described in detail in the priority U.S. Pat. No. 11,504,169 (which is incorporated herein in its entirety). The arrows 130 in FIG. 6 show that the first and second members 102, 104 can be moved toward each other (one member moving toward the other, or else both members moving toward each other) to cause the bending surfaces 122, 124 to press against the distal portion 16 of the shaft 14 in the bending zone 128 and thus bend shaft 14.

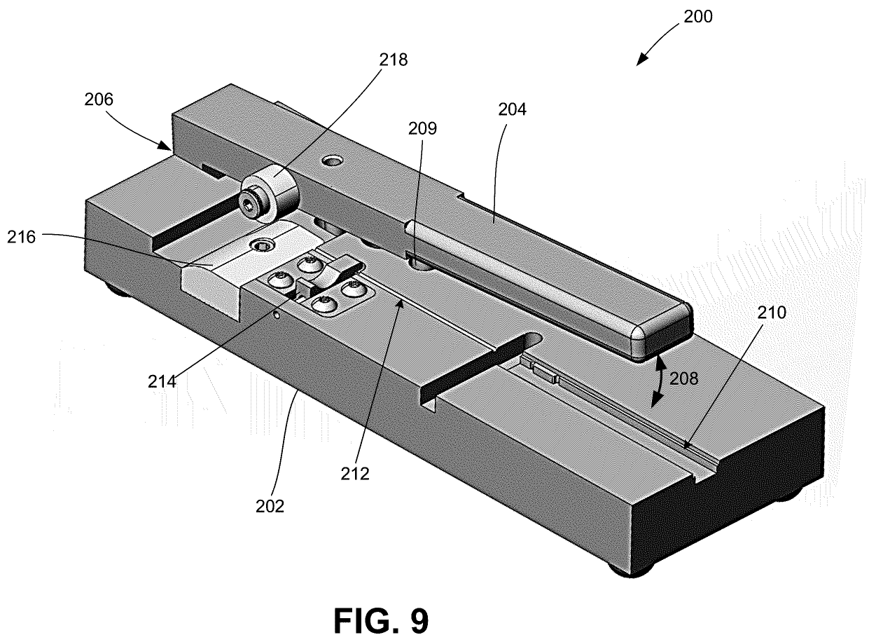

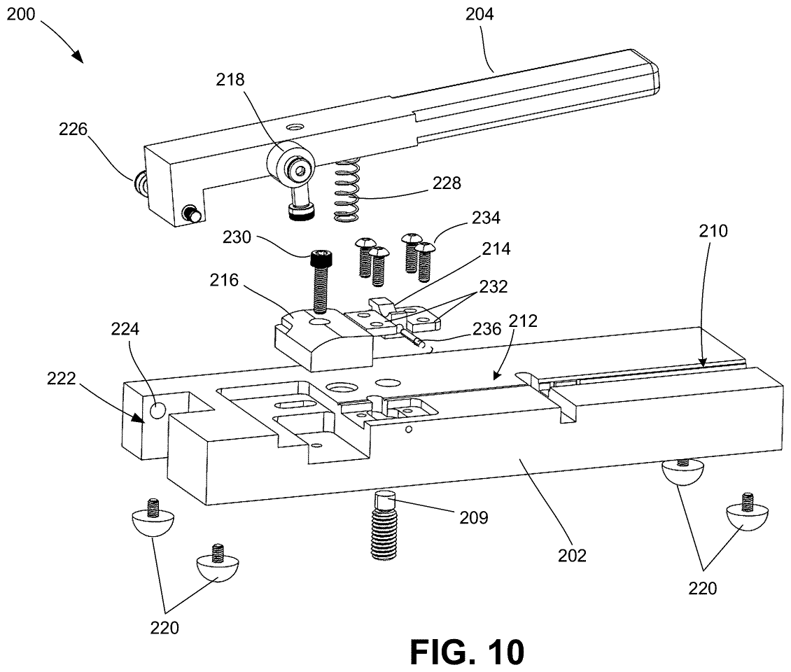

After the end-user tool 100 has been used by a person to bend the distal portion 16 of the shaft 14 of the depth gauge 10, the entirety of the depth gauge 10 can be removed from the end-user tool 100. As shown in FIGS. 7 and 8, the removed depth gauge 10 then will have a bend in the distal portion 16 of the shaft 14 of the probe 12. The angle θ of the bend typically will be acute (that is, less than 90 degrees), and, as shown in FIG. 8, it can be a relatively small deviation from the before-bending straight or substantially straight shaft 14. Longitudinal axis A is the axis along the length of the shaft 14 before its distal portion 16 is bent by the end-user tool 100, and axis or plane B shows the off-axis (with respect to the longitudinal axis A) angle θ that was created in the distal portion 16 of the shaft 14 by use of the end-user tool 100. The angle θ of the bend that is caused by use of the end-user tool 100 on the depth gauge 10 can be, for example, up to 10 degrees. One example is a bend of 1 degree, and other examples are bends of 3 degrees or 5 degrees. It is possible that an exact desired bend angle θ (such as 4 degrees) is not achievable with the end-user tool 100, and that use of the end-user tool 100 will result in a bend angle θ within a certain range (such as 2 to 3 degrees or 2 to 4 degrees). To achieve any non-zero acute angle θ bend (or bend angle range) in the distal portion 16 of the shaft 14 of the probe 12 of the depth gauge 10, the bending surfaces 122, 124 of the first and second members 102, 104 of the end-user tool 100 need to be configured accordingly. The bending surfaces 122, 124 may look different than shown in FIGS. 1, 2, 4, 5, and 6 if the target or desired bend angle θ (or bend angle range) is 5 degrees (or 4 to 6 degrees) versus 10 degrees (or 9 to 11 degrees).

The use of the end-user tool 100 to create a bend in the distal portion 16 of the shaft 14 of the probe 12 of the depth gauge 10, whatever that acute bend angle is, will give the depth gauge 10 a bent-probe feature that will allow an end user (such as a surgeon or a nurse) to feel very well the interior side wall(s) of a drilled hole in a bone of a patient (when the probe 12 is inserted into that hole and the depth gauge 10 is manually manipulated by the end user) and/or to feel very well when the distal end of the probe 12 emerges from the opposite end of the hole and catches on the lip of that opposite-side exit aperture of a through hole in the patient's bone. The user's tactile experience when using a bent-probe depth gauge in a medical procedure will be enhanced as compared to a depth gauge without a bent probe.

Having described the end-user tool 100 and how it is made and used, reference now is made to FIGS. 9-18 to describe the manufacturing tool 200 and how it is made and used.

FIG. 9 shows the manufacturing tool 200 that is designed to be placed on the top of a work surface within a manufacturing and/or assembly environment for use by a manufacturing and/or assembly person to bend the probe 12 of the depth gauge 10, before the finished bent-probe depth gauge is placed within sterile packaging for shipment to a hospital, surgery center, or other end-user destination.

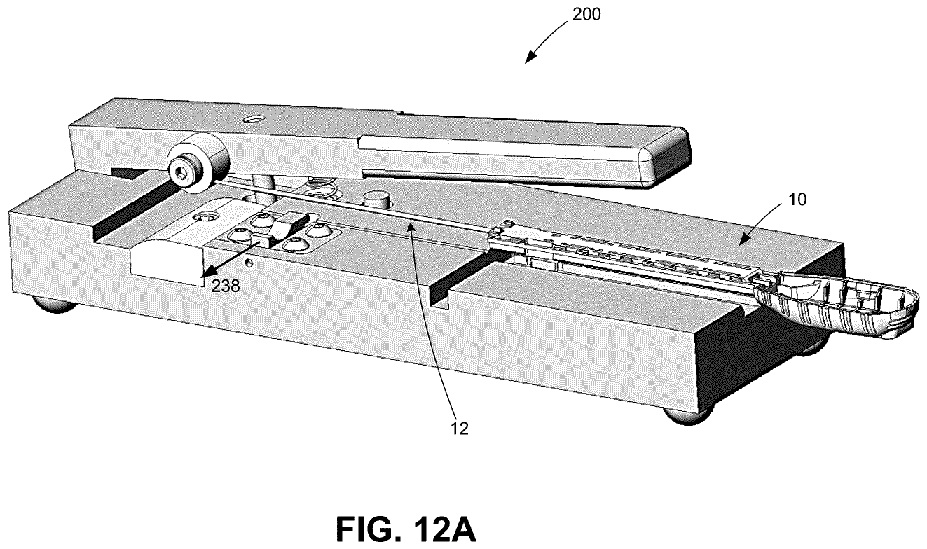

Referring to FIGS. 9 and 10, the manufacturing tool 200 comprises a base 202 configured to be disposed on the work surface. The base 202 can have feet 220 that contact the top of the work surface and can be made of rubber or other material that provides sufficient stability to the manufacturing tool 200 while in use by the manufacturing-assembly person. The base 202 defines a channel 212 for receiving at least a portion of the probe 12 of the depth gauge 10. The manufacturing tool 200 also comprises a lever 204 pivotally coupled to the base 202, by a pivot or hinge connection 206, to allow the lever 204 to move with respect to the base 202 (when, for example, the base 202 is disposed on the work surface). A bend is created in the distal portion 16 of the shaft 14 of the probe 12 of the depth gauge 10 when at least the distal portion 16 of the shaft 14 is received within the channel 212 and the lever 204 is moved toward the channel 210 of the base 202, as indicated by arrow 208. The channel 212 is the second of at least two channels defined by the base 202. A first channel 210 defined by the base 202 is for receiving at least a portion of the body or handle of the depth gauge 10.

The hinge connection 206 of the manufacturing tool 200 can include a hinge pin 226 that is disposed through the lever 204 and that is received within receiving pin holes 224 in the base 202, to allow the lever 204 to move with respect to the base 202. The pin holes 224 of the base 202 can be formed in a portion 222 of the base 202 that also receives a portion of the lever 204 through which the hinge pin 226 is disposed.

The base 202 of the manufacturing tool 200 can include a hard-stop feature 209 which can be a threaded bolt or plug that extends up from a top surface of the base 202 and is positioned to engage a bottom surface of the lever 204 when the lever 204 is moved by the manufacturing-assembly person down toward the base 202. This hard-stop feature 209 prevents the lever 204 from being moved down toward the base 202 any more than is needed for the manufacturing tool 200 to create the desired bend in the shaft 14 of the probe 12 of the depth gauge 10.

The base 202 of the manufacturing tool 200 also can include a sliding member 214 that provides a sliding lock feature, and the base 202 also can include an insert 216 that provides the necessary surface configuration to achieve a desired bend angle in the distal portion 16 of the shaft 14 of the probe 12. The sliding member 214 can be moved from an open position to a closed position. When in the open position, the shaft 14 of the probe 12 can be received within the second channel 212. When the sliding member 214 is in the closed position, the shaft 14 is held in place in the second channel 212.

The lever 204 of the manufacturing tool 200 can include a roller member 218 configured to cooperate with the insert 216 of the base 202. When the manufacturing-assembly person pushes down on the lever 204, with the shaft 14 of the probe 12 in the second channel 212, a bend is created in the shaft 14 at the location of the roller member 218 and the insert 216.

Each of the base 202 and the lever 204 of the manufacturing tool 200, as well as some or all of the parts of the base 202 and the lever 204 (such as the adjustable bolt 209, the sliding member 214, the insert 216, the roller member 218, and the hinge pin 226), can be formed of one or more metals and/or one or more alloys, such as, for example, steel (including, for example, stainless, hard, carbide, mild, high speed), cast iron, titanium, nickel-based alloys, cobalt-chrome alloy, and/or hardened aluminum.

Focusing now on FIG. 10, the manufacturing tool 200 also can include a spring 228, an insert securement screw 230, rails of wings 232, rails securement screws 234, and a spring-loaded pin 236. The spring 228 is for maintaining the lever 204 in a default position, which is up and away from the base 202. The insert securement screw 230 is for securing the insert 216 to the base 202. The pair of rails 232 are for receiving the four securement screws 234 so that the sliding member 214 can be placed into the base 202, and the spring-loaded pin 236 is for operably coupling the sliding member 214 in place and keeping it in its default/closed/locked position until the sliding member 214 is pulled back to allow the shaft 14 of the probe 12 to be placed into the second channel 212.

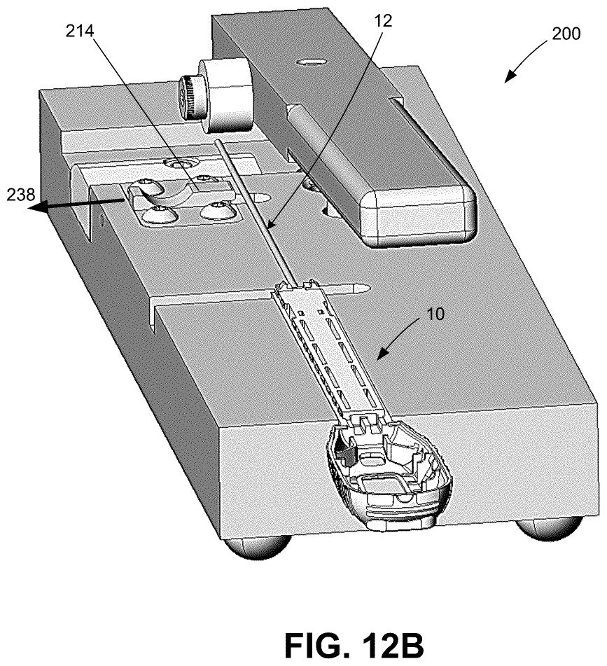

FIG. 11 for the manufacturing tool 200 and depth gauge 10 is similar to FIG. 4 with regard to the end-user tool 100. FIG. 11 shows the assembled manufacturing tool 200, waiting for the probe 12 of the depth gauge 10 to be placed in the second channel 212 of the base 202 and for at least a portion of the body or handle of the depth gauge 10 to be placed in the first channel 210 of the base 202.

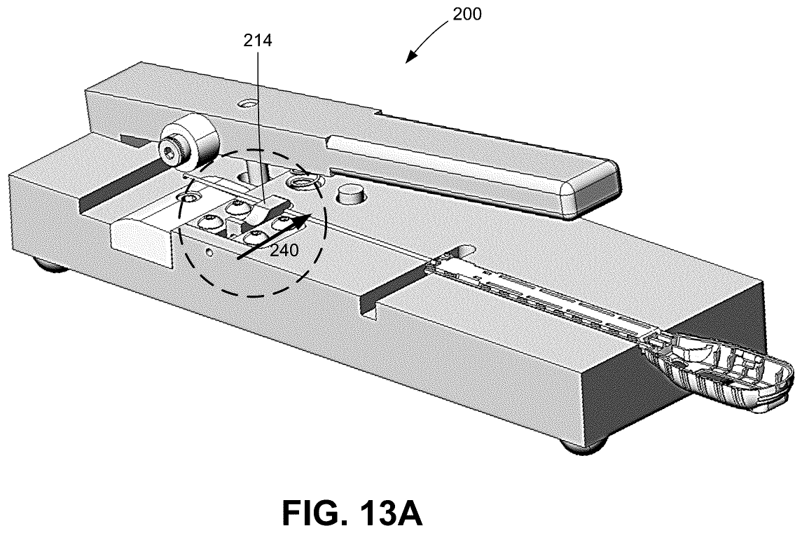

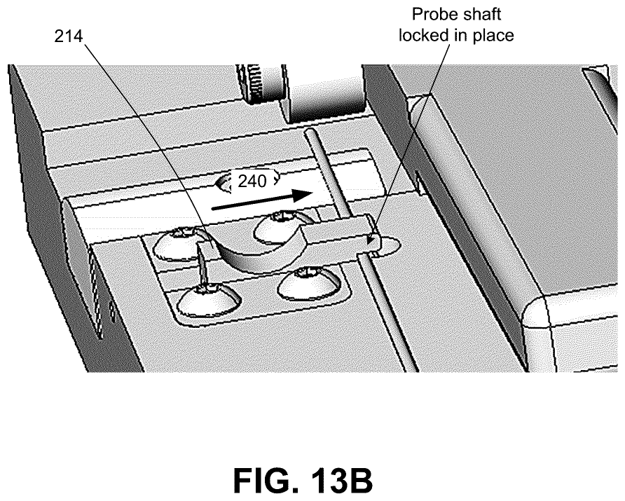

FIG. 12A shows the depth gauge 10 positioned over the base 202 of the manufacturing tool 200 and ready to be dropped into the first and second channels 210, 212 of the base 202, with the sliding member 214 pulled back (by a finger of the manufacturing-assembly person, not shown) in the direction of arrow 238 to put the sliding member into its open/unlocked position and thus to allow the probe 14 to be dropped or placed into the second channel 212 of the base 202. FIG. 12B shows the depth gauge 10 positioned within the channels 210, 212 of the base 202, with the sliding member 214 still pulled back into its open/unlocked position. FIGS. 13 A and B show how the release of the sliding member 214 results in it moving back to its default position which is the closed/locked position. The arrow 240 indicates the direction the sliding member 214 moves when it is released by the manufacturing-assembly person, to go into its locked/closed position and hold the probe 14 within the second channel 212. This also positions the distal portion 16 of the shaft 14 of the probe 12 over the insert 216 and ready for being contacted by the roller member 218 of the lever 204 when the lever 204 is pushed down toward the base 202 by the manufacturing-assembly person.

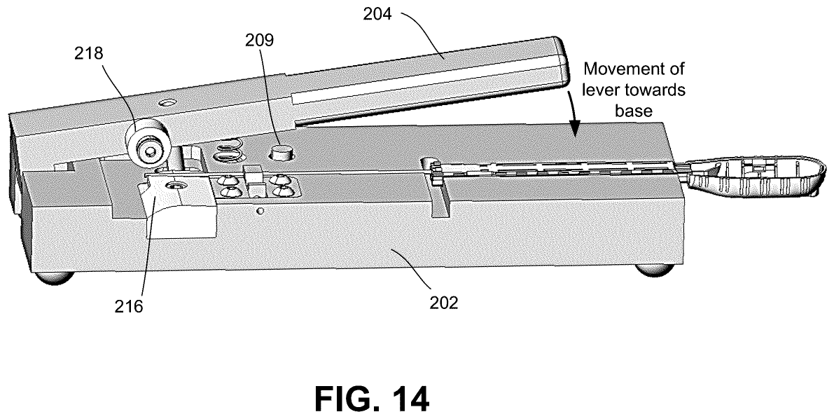

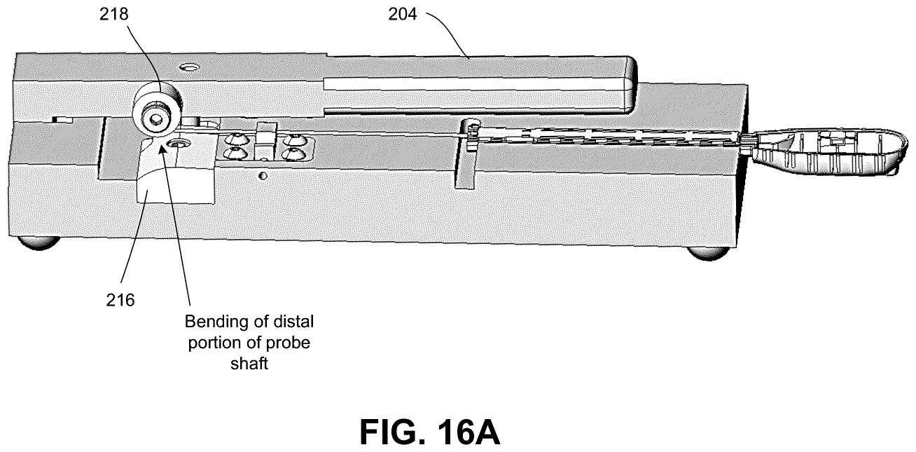

It is noted that in FIGS. 12A, 12B, and 13A (and also later in FIGS. 14, 16A, and 16C) the body or handle of the depth gauge 10 is shown as a half clam shell bottom piece without its other clam shell top piece. This is for clarity of how the body or handle of the depth gauge 10 is placed and received within the first channel 210 of the base 202 of the manufacturing tool 200, and it also is to convey how the probe 12 of the depth gauge 10 can be bent by use of the manufacturing tool 200 before the depth gauge 10 is fully assembled and ready for sterile packaging.

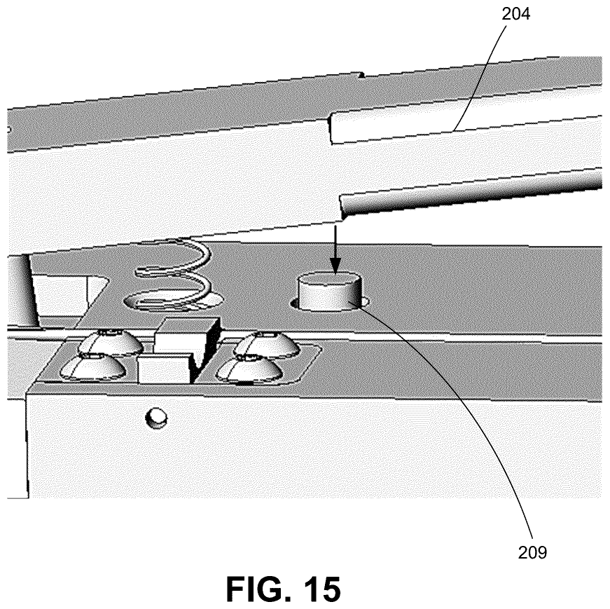

FIGS. 14 and 15 show how the manufacturing tool 200 with the depth gauge 10 fully received and loaded into it is now ready to be manipulated by the manufacturing-assembly person. The manufacturing-assembly person now can push down on the lever 204 to move the lever 204 toward the base 202, as indicated by the directional arrow shown in each of FIGS. 14 and 15. The lever 204 can only go down toward the base 202 as the hard-stop feature 209 of the base 202 will allow.

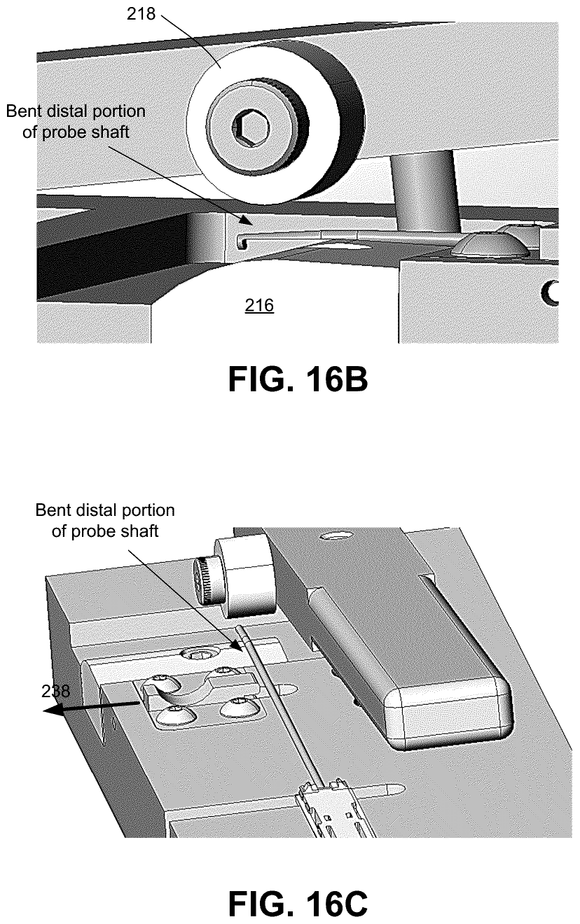

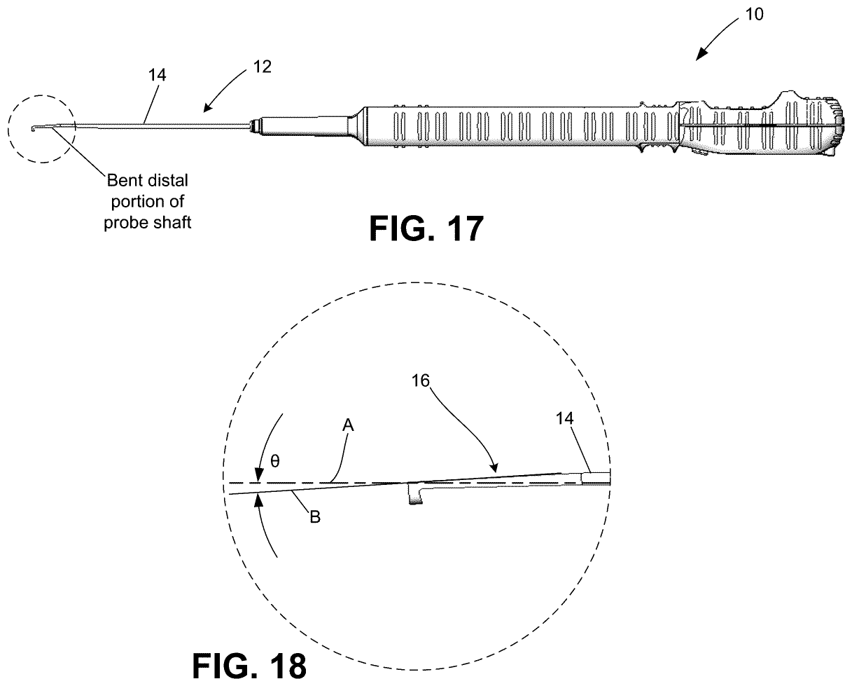

With the lever 204 pushed down toward the base 202 until it is stopped by the hard-stop feature 209, the distal portion 16 of the shaft 14 of the probe 12 of the depth gauge 10 is bent by being contacted from above by the roller member 218 of the lever 204 and from below by the insert 216 of the base 202, as shown in FIG. 16A. FIG. 16B shows the lever 204 moved up and away from the base 202 to reveal the bend in the probe 12, and FIG. 16C shows how the manufacturing-assembly person's finger (not shown) is then used on the sliding member 214 to move it back in the direction of the arrow 238 to open/unlock the sliding member 214 and thus allow the probe 12 to be removed from the second channel 212 of the base 202. With the bent-probe depth gauge 10 removed from the manufacturing tool 200, the bend in the distal portion 16 of the shaft 14 of the probe 12 can be seen more easily, as shown in FIGS. 17 and 18.

FIGS. 17 and 18 are with respect to the bend in the probe 12 that was created by the manufacturing tool 200, and these two drawings are similar to FIGS. 7 and 8 which show the bend in the probe 12 that was created by the end-user tool 100. The degree of bend in the distal portion 16 of the shaft 14 of the probe 12 that is achieved by the manufacturing-assembly person using the manufacturing tool 200 is similar to that achieved by a person using the end-user tool 100 on the probe 12 of the depth gauge 10, except that the manufacturing tool 200 is more complex in its construction and more precise in its ability to achieve a certain desired bend angle as compared to the ability of the end-user tool 100 to do so.

As shown in FIGS. 17 and 18, the removed depth gauge 10 will have a bend in the distal portion 16 of the shaft 14 of the probe 12 as indicated by the acute angle θ. The angle θ of the bend that is caused by use of the manufacturing tool 200 on the depth gauge 10 can be, for example, up to 15 degrees. One example is a bend of 2 degrees, and other examples are bends of 4 degrees or 6 degrees. To achieve any non-zero acute angle θ bend in the distal portion 16 of the shaft 14 of the probe 12 of the depth gauge 10, the insert 216 and the roller member 218 of the manufacturing tool 200 need to be configured accordingly. The insert 216 and/or the roller member 218 may look different than shown in FIGS. 9-16 if the target or desired bend angle θ is 2 degrees versus 8 degrees, for example.

The use of the manufacturing tool 200 to create a bend in the distal portion 16 of the shaft 14 of the probe 12 of the depth gauge 10, whatever that acute bend angle is, will give the depth gauge 10 a bent-probe feature that will allow an end user (such as a surgeon or a nurse) to feel very well the interior side wall(s) of a drilled hole in a bone of a patient (when the probe 12 is inserted into that hole and the depth gauge 10 is manually manipulated by the end user) and/or to feel very well when the distal end of the probe 12 emerges from the opposite end of the hole and catches on the lip of that opposite-side exit aperture of a through hole in the patient's bone. The user's tactile experience when using a bent-probe depth gauge in a medical procedure will be enhanced as compared to a depth gauge without a bent probe.

The tools and related methods shown and described herein with reference to FIGS. 1-8 and to FIGS. 9-18 have various features and aspects, and these features and aspects can be combined in ways other than specifically disclosed herein. All such combinations are to be considered included herein even if not expressly stated or shown given that the invention is not limited only to the specific details or embodiments disclosed herein.

1. A device for use by an end user, before or during a medical procedure on a patient, to bend a probe of a depth gauge after the depth gauge is removed from sterile packaging, the device comprising:
a first member having a free end, a length, and a width, with the length of the first member being greater than the width of the first member;
a second member having a free end, a length, and a width, with the length of the second member being greater than the width of the second member, the first and second members disposed opposite each other with space between the first and second members, the space for receiving at least a portion of the probe of the depth gauge; and
a connector portion disposed opposite the free ends of the first and second members to connect the first and second members, the connector portion configured to allow the first and second members to move to increase or decrease the space between the first and second members, a bend being created in the probe at a location along a length of the portion of the probe received within the space when the end user causes the first and second members to move and decrease the space.

2. The device of claim 1 wherein the connector portion defines an aperture through which the portion of the probe of the depth gauge passes to occupy at least a portion of the space.

3. The device of claim 1 wherein the first member, second member, and connector portion are formed as a single piece by injection molding.

4. The device of claim 3 wherein the single piece comprises a thermoplastic material.

5. A device for bending a probe of a depth gauge before the depth gauge is sealed in sterile packaging for later removal from that packaging in connection with a medical procedure to be performed on a patient, the device comprising:
a base configured to be disposed on a work surface, the base defining a channel for receiving at least a portion of the probe of the depth gauge; and
a lever pivotally coupled to the base to allow the lever to move with respect to the base when the base is disposed on the work surface, a bend being created in the probe at a location along a length of the portion of the probe received within the channel when the lever is moved toward the channel of the base.

6. The device of claim 5 wherein the base also defines a different channel for receiving at least a portion of a body of the depth gauge.

7. The device of claim 5 further comprising a hinge pin that is disposed through the lever and that is received within the base to allow the lever to move with respect to the base.

8. The device of claim 5 wherein the base includes a sliding member that is movable from an open position to a closed position, the portion of the probe being able to be received within the channel when the sliding member is in the open position and the portion of the probe being held in place in the channel when the sliding member is in the closed position.

9. The device of claim 5 further comprising four feet disposed on a bottom surface of the base for contacting the work surface when the base is disposed on the work surface.

10. The device of claim 5 wherein the base includes a form member having a curved surface for creating the bend in the probe when the lever is moved toward the channel of the base.

11. The device of claim 10 wherein the lever includes a roller member for pushing on the location along the length of the portion of the probe received within the channel to cause the bend to occur at that location and according to the curved surface of the form member when the lever is moved toward the channel of the base.

12. The device of claim 5 wherein each of the base and lever is formed of one or more metals.

13. The device of claim 5 wherein each of the base and lever is formed of one or more alloys.

14. A method of bending a probe of a depth gauge, comprising:
disposing within a device at least a portion of the probe of the depth gauge;
operating the device to bend the probe at a location along a length of the portion of the probe disposed within the device; and
removing the portion of the probe from the device to allow the depth gauge with the bent probe to be packaged or used.
